(19) United States
(12) Patent Application Publication
(10) Pub. No.: US 2017/0135946 A1
(43) Pub. Date: May 18, 2017

(54) Moldable Composition for Keratin Fibers

(71) Applicant: Avon Products, Inc., Suffern, NY (US)

(72) Inventors: Candice DeLeo Novack, Monroe, NY (US); Alicia N. Potuck, Suffern, NY (US)

(21) Appl. No.: 14/943,121

(22) Filed: Nov. 17, 2015

(51) Int. Cl. A61K 8/92; A61K 8/90; A45D 2/48; A61K 8/34; A61K 8/891; A45D 7/04; A61K 8/87; A61Q 1/10

(52) U.S. Cl. CPC A61K 8/927; A61K 8/87; A61K 8/90; A61Q 1/10; A61K 8/342; A61K 8/891; A45D 7/04; A45D 2/48; A61K 2800/43; A61K 2800/594; A61K 2800/30; A61K 2800/49; A61K 2800/524; A61K 2800/48

(57) ABSTRACT

The present invention relates generally to compositions and methods of using them to treat keratin fibers. More particularly, the compositions according to the invention, such as mascaras and other cosmetics, comprise gelling agents and soft waxes, and are useful for imparting moldability to treated keratin fibers.

### FIELD OF INVENTION

The present invention relates generally to compositions and methods of using them to treat keratin fibers. More particularly, the compositions according to the invention, such as mascaras and other cosmetics, comprise gelling agents and soft waxes, and are useful for imparting moldability to treated keratin fibers.

### BACKGROUND OF THE INVENTION

Conventional mascaras based on hard waxes and polymeric film formers are known to provide color and shine and may act to set lashes in a desired configuration. However, once the configuration of the lashes is set, for example, upon curing of the film or evaporation of volatiles, the treated lashes lack sufficient plasticity to undergo further styling. Consequently, the mascara film may become brittle and be subject to flaking on deformation of the lashes and extended wear. Lacking in the art are mascaras and other cosmetics for treating keratin fibers, which are highly plastic and permit the lashes to be shaped or styled after the film has set and without substantial loss of the film. It is therefore an object of the invention to provide compositions and methods for treating keratin fibers which, subsequent to curing or drying on the surface of keratin fibers, permit the treated fibers to be molded and therefore remolded into desired styles.

### SUMMARY OF THE INVENTION

Compositions and methods for styling keratin fibers (e.g., eyelashes) are provided. The compositions are characterized by an ability to deform plastically in the dry or cured state and to substantially retain the deformed configuration after an applied force is removed. The plastic deformation is reversible such that the compositions are capable of reverting to the original configuration on application of a subsequent force. This permits the treated lashes to be molded and thereafter re-molded through a range of configurations.

In one aspect of the invention, compositions (e.g., pigmented compositions, such as mascaras) are provided, typically comprising an oil, a polymeric gellant for forming a gel with the oil, and a soft wax. The soft wax is typically one having a melting point below 75° C., or below 72.5° C., or below 70° C. The oil (e.g., a hydrocarbon, ester oil, or fatty alcohol, including saturated or partially unsaturated C₆₋₃₀ or C₁₂₋₂₆ fatty alcohols and branched fatty alcohols such as octyldodoecanol) is typically one which is capable of forming a gel with the polymeric gellant. The oil may be present in an amount between about 0.1% and about 20% (e.g., between about 0.5-15%, between about 1%-10%, or between about 5%-8%) by weight of the composition. The polymeric gellant may comprise a mixed block copolymer. In some implementations, the copolymer has at least two distinct blocks of different polymeric α-olefins. The mixed block copolymer may, for example, be comprised of at least two, at least three, or more, blocks independently selected from different α-olefins, including ethylene, propylene, butylene, pentene, hexene, styrene, C₅₋₈ cyclo-olefins (e.g., cyclopentene) and the like. In one embodiment, the mixed block copolymer may be an ethylene mixed block copolymer comprising an ethylene block and at least one other α-olefin block, such as propylene and/or butylene. In one implementation, the block copolymer has the INCI name butylene/ethylene/propylene copolymer. The block copolymer may be present in an amount between about 0.01% and about 10% (e.g., between about 0.01%-5%, or between about 0.05%-1%, or between about 0.1%-0.5%) of the composition. The composition typically includes a low melting point wax, which may, for example, have a melting point below 75° C., or below 72.5° C., or below 70° C. In certain implementations, the low melting point wax will include one or more of paraffin wax, ozokerite, silicone wax, beeswax and modified beeswax derivatives such as bleached beeswax, sorbitol beeswax, and PEG-modified beeswax (e.g., PEG-8 beeswax and PG-3 beeswax). The low melting point wax may be present in an amount between about 5% and about 40% (e.g., between about 7.5%-30%, between about 10%-25%, or between about 15%-20%) by weight of the composition. In one embodiment, the composition also comprises a hard wax in combination with the low melting point wax. A hard wax may, for example, have a melting point above 70° C. (or above 72.5° C., or above 75° C., or above 80° C., or above 85° C., etc.). In some implementations, a hard wax is included in an amount less than about 5%, or less than about 3%, or less than about 2%, or less than about 1%, or less than about 0.5% by weight, including an amount between about 0.01% and about 3% by weight of the composition. In another embodiment, the composition may be substantially free (e.g., comprise less than 1%, or less than 0.1%, or less than 0.05%) of a hard wax, including, for example, one having a melting point above 75° C., or above 80° C., or above 85° C., etc. In other embodiments, the compositions are free of hard waxes, including waxes with melting points above 75° C. (or above 80° C., or above 85° C., etc.). The composition may also comprise a polymeric film former (e.g., polyacrylates, polyurethanes including polyurethane-35 for example, polyvinylpyrolidone, cellulosics, polydextrose, polysaccharides, polyimides, polyamides, etc.). In some embodiments, the polymeric film former is water-soluble and/or water-dispersible. In other embodiments, the polymeric film former is not water-soluble and/or not water-dispersible. If included, the film former is typically present in an amount between about 0.1% and about 30% (e.g., between about 1%-25%, or between about 2%-10%) by weight of the composition. The composition may also comprise one or more particulates (e.g., any suitable particulate fillers, lakes, or pigments), which may be present individually or in the aggregate in an amount between about 0.01% and about 30% (e.g., between about 0.1%-25%, between about 1%-20%, between about 3%-15%, or between about 5%-10%) by weight of the composition. In some implementations, the compositions will comprise one or both of carbon black and iron oxide, each of which may be present, individually or in the aggregate with all other particulates, in the foregoing amounts. In one embodiment, the composition may further comprise, in addition to the polymeric gellant, one or more amide-based oil-phase gellants, and in particular glutamide-based gellants, such as dibutyl laurolyl glutamide and/or dibutyl ethylhexanoyl glutamide, in an amount individually or collectively between about 0.001% and about 10% (e.g., between about 0.01%-5%, or between about 0.05%-1%, or between about 0.1%-0.5%) by weight of the composition. The compositions may comprise an aqueous phase, and may be in the form of emulsions (e.g., oil-in-water, water-in-oil, etc.), or they may be substantially anhydrous (e.g., <2%, for example, less than 1%, or less than 0.5%, or less than 0.25% water), or they may be anhydrous. The compositions may also include an aqueous phase rheology modifier, such as acrylates copolymer, in amounts sufficient to thicken the aqueous phase, typically from about 0.01% to about 20% by weight, more typically from about 0.1%-5% by weight of the composition.

In another aspect of the invention, methods of using the compositions to impart moldable films on keratin fibers (e.g., eyelashes) are provided. The films are capable of undergoing plastic, rather than elastic, deformation. The dried films are reversibly deformable such that they can be molded, and then remolded back into the original configuration, or molded into subsequent configurations. The dried compositions may be molded with application forces less than, for example, 60 grams. The dried films may be characterized by a force on compression of less than 65 grams (e.g., less than 60 grams, or less than 50 grams, or less than 40 grams, or less than 30 grams, or less than 20 grams) when displaced by 2 mm at a constant force of 2 grams. The methods generally comprise applying a composition of the invention (e.g., a pigmented cosmetic such as a mascara) to keratin fibers to cover at least a substantial portion, or at least a major portion thereof. Upon evaporation of volatiles (e.g., partial or complete evaporation of solvents and other volatiles), the composition forms a dry film on the surfaces of the keratin fibers that is readily moldable. For example, the treated lashes may be bent, curled, or crimped by applying a force thereto. Upon removal of the force, the treated lashes will remain substantially in the bent, curled, or crimped configuration. The keratin fibers may be molded into a first configuration (e.g., curled, straight, etc.), by applying a force to the treated fibers (e.g., by pressing, brushing, crimping, bending, etc.). After the force is removed, the keratin fibers remain substantially in the first configuration due to the fact that the film behaves plastically and non-elastically at the applied level of force. The treated keratin fibers may be subsequently re-molded into a second configuration by applying a second force to the fibers. After the second force is removed, the keratin fibers remain substantially in the second desired configuration.

These and other aspects of the present invention will become apparent to those skilled in the art according to the present description, including the figures and appended claims.

### DETAILED DESCRIPTION

As used herein, the term “consisting essentially of” is intended to limit the invention to the specified materials or steps and those materials or steps that do not materially affect the basic and novel characteristics of the claimed invention, for example, moldability, deformability, and/or layerability of a cured or dried film as understood from a reading of this specification.

As used herein, a film is “set” after it has been applied to an integument, when all chemical and physical processes necessary to achieve a suitable cosmetic film of sufficient transfer resistance, hardness, and substantivity have occurred, including, without limitation, partial or complete evaporation of solvents, including volatile solvents, cross-linking of any reactive polymers, formation of long-range order, e.g., gel structures, hydrogen-bonding networks, and the like. A film may “set” immediately on application if none of these physical or chemical changes are necessary for the ordinary wear of the cosmetics.

The terms “a” and “an,” as used herein and in the appended claims, mean “one or more” unless otherwise indicated. It should be noted that unless otherwise indicated, percent (%) is % by weight, based on the total weight of the composition (including any solvents or vehicle). It will be understood that the weight % of all components, in the aggregate, will not exceed 100%. Unless otherwise indicated, each component may be included in the compositions in amounts ranging from about 0.0001% by weight to about 20% by weight (e.g., 0.001-10% by weight). Any solvents or other vehicle used in the compositions of the invention are topically acceptable, by which is meant non-toxic and substantially non-irritating to human integuments.

The compositions of the invention are intended for application to any human integument, including skin, nails, lips, hair, lashes, etc. In some embodiments, the compositions are intended for styling keratin fibers, (e.g., eyelashes, hair of the scalp, etc.), most notably eyelashes. The compositions of the invention, such as pigmented mascaras, are generally characterized by a unique ability to plastically deform once they have been applied to keratin fibers and allowed to dry or partially dry (e.g., after evaporation or partial evaporation of volatiles). The film is pliable and readily moldable due to its plastic, non-elastic nature and is consequently resistant to flaking and cracking. This permits the keratin fibers to be molded and re-molded through a range of configurations that may be desired by a user. Following multiple applications and extended wear of the inventive compositions, the films remain moldable without substantial loss of the film caused by brittleness and flaking.

The compositions of the invention typically comprise an oil, a polymeric gellant capable of forming a gel with the oil, and a soft wax. As used herein, a “soft wax” is one having a melting point below 75° C., but more typically below 72.5° C., or below 70° C.

The polymeric gellant is capable of gelling or structuring an oil phase. It typically comprises a block copolymer, for example, having at least two distinct blocks. The blocks may each comprise different polymeric α-olefins. The mixed block copolymer may, for example, be comprised of at least two, at least three, or more, blocks. The blocks may, for example, be independently selected from C₂-C₁₀ or C₂-C₈ or C₂-C₆ or C₂-C₄ olefins, including without limitation, ethylene, propylene, butylene, pentene, hexene, styrene, and C₅₋₈ cyclo-olefins (e.g., cyclopentene). In one embodiment, at least one, or at least two blocks comprise an olefin independently selected from those of the form H₂C═CH₂—R, where R is hydrogen, halogen, hydroxyl, or a C₁-C₁₀hydrocarbon (e.g., alkyl, alkenyl, alkynyl, aryl, heteroaryl, and combinations thereof), optionally substituted with 1-6 heteroatoms selected from oxygen, nitrogen, sulfur, and halogen, or perfluorinated derivatives thereof. In one embodiment, the mixed block copolymer is an ethylene mixed block copolymer comprising an ethylene block and at least one other block, such as an α-olefin block, for example propylene and/or butylene.

In one embodiment, the block copolymer is butylene/ethylene/propylene copolymer (INCI), such as GEL BASE, sold by Lonza. A polymeric gellant “consisting essentially of” a particular mixed block copolymer, including butylene/ethylene/propylene copolymer, is intended to mean that the presence of additional polymeric gellants, including mixed block copolymers, in amounts which would measurably affect the moldability, deformability, and/or layerability of the composition are excluded.

The block copolymer will typically be present in an amount sufficient to impart structure to the composition (i.e., to have a measurable impact on viscosity, for example, ±5% or more). The block copolymer may be present in an amount sufficient to provide a viscosity to the composition of at least 1,000 cps, at least 2,500 cps, at least 5,000 cps, at least 10,000 cps, at least 25,000 cps, at least 50,000 cps, at least 100,000 cps, or at least 200,000 cps at 25° C. In some embodiments, the block copolymer gellant will be present in an amount from about 0.001 to about 10%, from about 0.01 to about 5%, from about 0.1 to about 5%, or from 0.1% to about 1%, by weight of the composition. In some specific embodiments, the block copolymer is present in an amount of about 0.1%, about 0.2%, about 0.3%, about 0.4%, about 0.5%, about 0.6%, about 0.7%, about 0.8%, about 0.9%, or about 1.0% by weight of the composition.

The composition may also comprise additional oil phase and/or aqueous phase gellants. In one embodiment, the composition will comprise one or more glutamide-based oil-phase gellants, such as dibutyl lauryl glutamide and/or dibutyl ethylhexanoyl glutamide and combinations thereof. In some embodiments the composition may comprise dibutyl lauroyl glutamide. In other embodiments the composition may comprise dibutyl ethylhexanoyl glutamide. Dibutyl lauroyl glutamide and dibutyl ethylhexanoyl glutamide (are available from Ajinomoto Co., Inc. as GP-1 and EB-21, respectively.

In another embodiment, the composition may comprise as an oil-phase gellant, a polyamide gelling agent, such as an ester-terminated polyester amide (ETPEA). One suitable ETPEA gellant is Sylvaclear C75 (Arizona Chemicals). Sylvaclear C75 and additional suitable gellants and solvents therefore are described in U.S. Pat. No. 7,989,002, the entire contents of which are hereby incorporated by reference. Also suitable are the gellants and solvents therefore as described in U.S. Pat. No. 7,682,621, the entire contents of which are hereby incorporated by reference. Additional oil-phase and aqueous-phase gellants may be included, individually or in the aggregate in an amount from about 0.001% to about 20%, or from about 0.01% to about 10% by weight of the composition.

The composition typically includes one or more soft waxes. As used herein, a “soft wax” is one having a melting point below 75° C. The melting values provided herein refer to the mid-point of the melting range. In some embodiments, the soft waxes will have an onset of melting temperature below 75° C. Each of the melting values disclosed herein may refer to the midpoint of the melting range, onset of the melting range, or point at which the wax is completely melted.

Table 1 provides representative suitable soft waxes arranged by approximate melting point or melting range.

TABLE 1

| Wax | Melting Point (° C.) |
|---|---|
| esparto wax | 73 |
| ozokerite wax | 72 |
| jojoba wax | 70 |
| candelilla wax | 68-73 |
| ceresin wax | 67-71 |
| beeswax | 62-64 |
| castor wax | 60 |
| sugarcane wax | 60 |
| stearyl alcohol | 59 |
| hard tallow | 57-60 |
| cetyl alcohol | 56 |
| petrolatum | 54 |
| glyceryl monostearate | 54-56 |
| Japan wax | 53 |
| silicone waxes | 53-75 |
| paraffin wax | 50-60 |
| lanolin alcohol | 45-60 |
| bayberry wax | 45 |
| cetyl palmitate | 43-53 |
| lanolin | 38-42 |
| illipe butter | 34-38 |
| cocoa butter | 31-35 |

In one embodiment, the composition comprises one or more hard waxes in combination with the one or more soft waxes. A hard wax may, for example, have a melting point above 75° C., or above 80° C., or above 85° C., or above 90° C., or above 95° C.

Table 2 provides representative suitable hard waxes arranged by approximate melting point or melting range.

TABLE 2

| Wax | Melting Point (° C.) |
|---|---|
| acrawax | 140 |
| microcrystalline petroleum wax | 99 |
| linear polyethylene wax | 95 |
| stearone | 89 |
| castor wax | 86 |
| montan wax | 82-95 |
| lignite wax | 82-95 |
| ouricouri wax | 81-84 |
| carnauba wax | 78-85 |
| rice bran wax | 77-86 |
| shellac wax | 74-78 |

It will be understood that the melting points and ranges provided in Table 1 and Table 2 are merely representative of typical values for each wax, and wide variation in the melting point or melting point range may be observed from sample to sample depending on the source and purity of the wax. It is within the skill in the art to determine the melting point or melting point range of any given wax sample. Melting points may be determined, for example, by DSC, or by drop melting point according to ASTM D127, incorporated by reference herein, and/or ring-and-ball softening point according to ASTM D36, incorporated by reference herein. In the event of a discrepancy between the techniques, melting point will be determined by DSC.

Because some waxes may have broad melting ranges, they may be considered as suitable soft waxes or suitable hard waxes, depending on the particular composition and the desired viscosity. For example, ozokerite may be considered a soft wax in the practice of the invention, but because it is toward the high end of the melting point range for soft waxes, in some embodiments, the composition will be substantially free of ozokerite, by which is meant it is present in amounts insufficient to measurably contribute to the viscosity of the composition (e.g., comprises less than 1%, or less than 0.1% or less than 0.01% by weight). Similarly, although shellac wax may be considered a hard wax based on Table 2, because it has a broad melting point range, it may also be useful as a soft wax in some embodiments.

The waxes may be natural, mineral and/or synthetic waxes. Natural waxes are those of animal origin, including without limitation beeswax, spermaceti, lanolin, and shellac wax, and those of vegetable origin, including without limitation carnauba, candelilla, bayberry, and sugarcane wax.

Mineral waxes contemplated to be useful include, without limitation ozokerite, ceresin, montan, paraffin, microcrystalline, petroleum, and petrolatum waxes.

Synthetic waxes include, for example, polyethylene glycols such as PEG-18, PEG-20, PEG-32, PEG-75, PEG-90, PEG-100, and PEG-180 which are sold under the tradename CARBOWAX® (The Dow Chemical Company). Mention may be made of CARBOWAX 1000 which has a molecular weight range of 950 to 1,050 and a melting point of about 38° C., CARBOWAX 1450 which has a molecular weight range of about 1,305 to 1,595 and a melting point of about 56° C., CARBOWAX 3350 which has a molecular weight range of 3,015 to 3,685 and a melting point of about 56° C., and CARBOWAX 8000 which has a molecular weight range of 7,000 to 9,000 and a melting point of about 61° C.

Synthetic waxes also include Fischer Tropsch (FT) waxes and polyolefin waxes, such as ethylene homopolymers, ethylene-propylene copolymers, and ethylene-hexene copolymers. Representative ethylene homopolymer waxes are commercially available under the tradename POLYWAX® Polyethylene (Baker Hughes Incorporated) with melting points ranging from 80° C. to 132° C. Commercially available ethylene-α-olefin copolymer waxes include those sold under the tradename PETROLITE®. Copolymers (Baker Hughes Incorporated) with melting points ranging from 95° C. to 115° C.

In some embodiments, the composition may comprise one or more waxes selected from the group consisting of paraffin wax, ozokerite, silicone wax, beeswax and beeswax derivatives such as bleached beeswax, sorbitol beeswax, and PEG-modified beeswax, PEG-8 beeswax and PG-3 beeswax, and silicone waxes, such as SILWAX CRM2, SILWAX 5022, SILWAX L118, SILWAX D221M, and SILWAX Di-5026. These waxes may be present, individually or in the aggregate, in an amount from about 0.1%-40% by weight, or 0.5%-25% by weight, or 1%-15% by weight of the composition.

In one embodiment, the composition is free of, or substantially free of jojoba wax. In another embodiment, the composition is free of, or substantially free of ozokerite wax. In another embodiment, the composition is free of, or substantially free of candelilla wax. In another embodiment, the composition is free of, or substantially free of ceresin wax. In another embodiment, the composition is free of, or substantially free of carnauba wax. As used herein, “substantially free” means that the wax is present in amounts insufficient to measurably contribute to the viscosity of the composition (e.g., comprises less than 1%, or less than 0.1% or less than 0.01% by weight).

The soft waxes, either individually or in the aggregate, may be present in the compositions in an amount between about 0.01% to about 40%, about 0.1% to about 35%, about 1% to about 30%, about 2.5% to about 25%, or about 5% to about 20% by weight of the composition. In some embodiments, the soft wax component may comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of the composition.

In some embodiments, the composition optionally comprises one or more hard waxes. If present, hard waxes will be present individually or in the aggregate, in an amount between about 0.01% and about 5%, between about 0.1% and about 3%, between about 0.5% and about 2.5%, or between about 1% and about 2% by weight of the composition. In another embodiment, the composition may be “substantially free” of a hard wax, by which is meant less than 1% (w/w). In other embodiments, the compositions are free of hard waxes. As used herein, hard waxes have a melting point above 75° C.

The soft and/or hard wax component may include one or more low opacity waxes (e.g., a ΔL* less than 8 as determined by the procedure set forth below. The hard and soft waxes collectively may have a ΔL* value of less than 8 and/or each individual wax may have a ΔL* value of less than 8. In other embodiments the ΔL* value of the waxes, individually is 10 or less, 9 or less, 8 or less, 6 or less, 5 or less, 4 or less, 3 or less, 2 or less, or 1 or less. In certain embodiments the wax component may comprise one or more individual waxes having a ΔL* less than 8, in combination with one or more waxes individually having a ΔL* of 8 or greater, as long as in the aggregate, the combination of waxes (i.e., the wax component) exhibits a ΔL* less than 8. In one embodiment, the wax component does not comprise an individual wax having a ΔL* of 8 or greater. In other embodiments the wax component does not comprise more than 15%, more than 10%, or more than 5%, or more than 1% of a wax having a ΔL* value of 8 or greater, by weight of the wax component.

ΔL* is measured by measuring L* values on a drawdown film on a black Leneta card using a hand-held spectrophotometer (e.g., a Konica Minolta CM-2600d spectrophotometer). The drawdown film is obtained by applying 3 mL of the sample to obtain a test film on the Leneta card that is about 75 microns in thickness and allowed to dry for 2 hours. The Leneta card itself is the standard for the color black in the tristimulus color measurement method, and by definition has an L value of zero. The ΔL* of the entire composition may be measured using the same protocol.

Suitable low opacity waxes include, but are not limited to, carnauba wax, beeswax, bleached beeswax, ozokerite, kahlwax 7307, and silicone waxes (e.g., SILWAX CRM2, SILWAX 5022, SILWAX L118, SILWAX D221M, SILWAX Di-5026), POE (20M) sorbitol beeswax, PEG-8 beeswax, and other modified beeswax derivatives, variants and combinations thereof.

If included, the low opacity wax may be present individually or in the aggregate, in an amount between about 0.01% and about 25%, between about 1% and about 20%, or between about 5% and about 10% by weight of the composition.

The composition typically comprises one or more oils that are capable of forming a gel with the polymeric gellant and/or with an optional additional oil-phase gellant. Suitable oils include, without limitation, vegetable oils; esters including emollient esters, such as octyl palmitate, isopropyl myristate and isopropyl palmitate; ethers such as dicapryl ether; fatty alcohols such as cetyl alcohol, and branched alcohols like octyldodecanol, stearyl alcohol and behenyl alcohol; isoparaffins such as isooctane, isododecane and isohexadecane; silicone oils such as dimethicones, cyclic silicones, and polysiloxanes; hydrocarbon oils such as mineral oil, petrolatum, isoeicosane and polyisobutene; and the like. Suitable hydrophobic hydrocarbon oils may be saturated or unsaturated, have an aliphatic character and be straight or branched chained or contain alicyclic or aromatic rings. The oil may be composed of a singular oil or mixtures of different oils.

Exemplary hydrocarbon oils may comprise straight or branched chain paraffinic hydrocarbons having from 5 to 80 carbon atoms, typically from 8 to 40 carbon atoms, and more typically from 10 to 16 carbon atoms, including but not limited to, pentane, hexane, heptane, octane, nonane, decane, undecane, dodecane, tetradecane, tridecane, and the like. Some useful hydrocarbon oils are highly branched aliphatic hydrocarbons, including C₈₋₉ isoparaffins, C₉₋₁₁ isoparaffins, C₁₂ isoparaffin, C₂₀₋₄₀ isoparaffins and the like. Special mention may be made of the isoparaffins having the INCI names isohexadecane, isoeicosane, and isododecane (IDD).

Paraffinic hydrocarbons are available from Exxon under the ISOPARS trademark, and from the Permethyl Corporation. In addition, C₈₋₂₀ paraffinic hydrocarbons such as C₁₂ isoparaffin (isododecane) manufactured by the Permethyl Corporation having the tradename PERMETHYL 99 A™ are also contemplated to be suitable. Various commercially available C₁₆ isoparaffins, such as isohexadecane (having the tradename PERMETHYL®) are also suitable. Examples of volatile hydrocarbons include polydecanes such as isododecane and isodecane, including for example, PERMETHYL-99A (Presperse Inc.) and the C₇-C₈ through C₁₂-C₁₅ isoparaffins such as the Isopar Series available from Exxon Chemicals.

Also suitable as hydrocarbon oils are poly-alpha-olefins, typically having greater than 20 carbon atoms, including (optionally hydrogenated) C₂₄₋₂₈ olefins, C₃₀₋₄₅ olefins, polyisobutene, hydrogenated polyisobutene, hydrogenated polydecene, polybutene, hydrogenated polycyclopentane, mineral oil, pentahydrosqualene, squalene, squalane, and the like. The hydrocarbon oil may also comprise higher fatty alcohols, such as oleyl alcohol, octyldodecanol, and the like.

Suitable oils may also comprise one or more volatile and/or non-volatile silicone oils. Volatile silicones include cyclic and linear volatile dimethylsiloxane silicones. In one embodiment, the volatile silicones may include cyclodimethicones, including tetramer (D₄), pentamer (D₅), and hexamer (D₆) cyclomethicones, or mixtures thereof. Particular mention may be made of the volatile cyclomethicone-hexamethyl cyclotrisiloxane, octamethyl-cyclotetrasiloxane, and decamethyl-cyclopentasiloxane. Suitable dimethicones are available from Dow Corning under the name DOW CORNING 200® Fluid and have viscosities ranging from 0.65 to 600,000 centistokes or higher. Suitable non-polar, volatile liquid silicone oils are disclosed in U.S. Pat. No. 4,781,917, herein incorporated by reference in its entirety. Additional volatile silicones materials are described in Todd et al., “Volatile Silicone Fluids for Cosmetics,” Cosmetics and Toiletries, 91:27-32 (1976), herein incorporated by reference in its entirety. Linear volatile silicones generally have a viscosity of less than about 5 centistokes at 25° C., whereas the cyclic silicones have viscosities of less than about 10 centistokes at 25° C. Examples of volatile silicones of varying viscosities include DOW CORNING 200, DOW CORNING 244, DOW CORNING 245, DOW CORNING 344, and DOW CORNING 345, (Dow Corning Corp.); SF-1204 and SF-1202 Silicone Fluids (G.E. Silicones), GE 7207 and 7158 (General Electric Co.); and SWS-03314 (SWS Silicones Corp.). Linear, volatile silicones include low molecular weight polydimethylsiloxane compounds such as hexamethyldisiloxane, octamethyltrisiloxane, decamethyltetrasiloxane, and dodecamethylpentasiloxane, to name a few.

Non-volatile silicone oils will typically comprise polyalkylsiloxanes, polyarylsiloxanes, polyalkylarylsiloxanes, or mixtures thereof. Polydimethylsiloxanes are non-volatile silicone oils. The non-volatile silicone oils will typically have a viscosity from about 10 to about 60,000 centistokes at 25° C., in one embodiment between about 10 and about 10,000 centistokes, and in one embodiment still between about 10 and about 500 centistokes; and a boiling point greater than 250° C. at atmospheric pressure. Non limiting examples include dimethyl polysiloxane (dimethicone), phenyl trimethicone, and diphenyldimethicone. The volatile and non-volatile silicone oils may optionally be substituted with various functional groups such as alkyl, aryl, amine groups, vinyl, hydroxyl, haloalkyl groups, alkylaryl groups, and acrylate groups, to name a few.

In one embodiment, the silicone oil may be a fluorinated silicone, such as a perfluorinated silicone (i.e., fluorosilicones). Fluorosilicones are advantageously both hydrophobic and oleophobic and thus contribute to a desirable slip and feel of the product. Fluorosilicones can be gelled with behenyl behenate if desired. One suitable fluorosilicone is a fluorinated organofunctional silicone fluid having the INCI name Perfluorononyl Dimethicone. Perfluorononyl Dimethicone is commercially available from Phoenix Chemical under the trade name PECOSIL®.

Additional suitable oils may include, for example, isostearyl neopentanoate, isostearyl stearate, castor oil, lauryl lactate, isopropyl palmitate, glyceryl triacethyl hydroxystearate, diisopropyl adipate, octyl isononanoate, neopentyl glycol dioctanoate, neopentyl glycol dicaprate, isodecyl oleate, and myristyl myristate.

The compositions may comprise one or more ester oils. The esters may be, for example, mono-esters, di-esters, or tri-esters. Ideally, the additional esters, if present, also provide emolliency to the composition.

Other suitable additional ester oils that may used in the compositions of the invention include fatty acid esters, and in particular, those esters commonly used as emollients in cosmetic formulations. Such esters will typically be the esterification product of an acid of the form R₄(COOH)₁₋₂ with an alcohol of the form R₅(OH)₁₋₃ where R₄ and R₅ are each independently linear, branched, or cyclic hydrocarbon groups, optionally containing unsaturated bonds (e.g., from 1-6 or 1-3 or 1), and having from 1 to 30 (e.g., 6-30 or 8-30, or 12-30, or 16-30) carbon atoms, optionally substituted with one or more functionalities including hydroxyl, oxa, oxo, and the like. Preferably, at least one of R₄ and R₅ comprises at least 8, or at least 10, or at least 12, or at least 16 or at least 18 carbon atoms, such that the ester oil comprises at least one fatty chain. The esters defined above will include, without limitation, the esters of mono-acids with mono-alcohols, mono-acids with diols and triols, di-acids with mono-alcohols, and tri-acids with mono-alcohols.

Suitable fatty acid esters include, without limitation, butyl isostearate, butyl oleate, butyl octyl oleate, cetyl palmitate, cetyl octanoate, cetyl laurate, cetyl lactate, cetyl isononanoate, cetyl stearate, diisostearyl fumarate, diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisopropyl dimerate, triisostearyl trilinoleate, octodecyl stearoyl stearate, hexyl laurate, hexadecyl isostearate, hexydecyl laurate, hexyldecyl octanoate, hexyldecyl oleate, hexyldecyl palmitate, hexyldecyl stearate, isononyl isononanaote, isostearyl isononate, isohexyl neopentanoate, isohexadecyl stearate, isopropyl isostearate, n-propyl myristate, isopropyl myristate, n-propyl palmitate, isopropyl palmitate, hexacosanyl palmitate, lauryl lactate, octacosanyl palmitate, propylene glycol monolaurate, triacontanyl palmitate, dotriacontanyl palmitate, tetratriacontanyl palmitate, hexacosanyl stearate, octacosanyl stearate, triacontanyl stearate, dotriacontanyl stearate, stearyl lactate, stearyl octanoate, stearyl heptanoate, stearyl stearate, tetratriacontanyl stearate, triarachidin, tributyl citrate, triisostearyl citrate, tri-C₁₂₋₁₃-alkyl citrate, tricaprylin, tricaprylyl citrate, tridecyl behenate, trioctyldodecyl citrate, tridecyl cocoate, tridecyl isononanoate, glyceryl monoricinoleate, 2-octyldecyl palmitate, 2-octyldodecyl myristate or lactate, di(2-ethylhexyl)succinate, tocopheryl acetate, and the like.

Other suitable esters include those wherein R₅ comprises a polyglycol of the form H—(O—CHR*—CHR*)ₙ— wherein R* is independently selected from hydrogen or straight chain C₁₋₁₂ alkyl, including methyl and ethyl, as exemplified by polyethylene glycol monolaurate.

Salicylates and benzoates are also contemplated to be useful esters in the compositions of the invention. Suitable salicylates and benzoates include esters of salicylic acid or benzoic acid with an alcohol of the form R₆OH where R₆ is a linear, branched, or cyclic hydrocarbon group, optionally containing unsaturated bonds (e.g., one, two, or three unsaturated bonds), and having from 1 to 30 carbon atoms, typically from 6 to 22 carbon atoms, and more typically from 12 to 15 carbon atoms. Suitable salicylates include, for example, octyl salicylate and hexyldodecyl salicylate, and benzoate esters including C₁₂₋₁₅ alkyl benzoate, isostearyl benzoate, hexyldecyl benzoate, benzyl benzoate, and the like.

Additional suitable esters include, without limitation, polyglyceryl diisostearate/IPDI copolymer, triisostearoyl polyglyceryl-3 dimer dilinoleate, polyglycerol esters of fatty acids, and lanolin, to name but a few.

Other suitable oils include, without limitation, castor oil, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, avocado oil, camellia oil, macadamia nut oil, turtle oil, mink oil, soybean oil, grape seed oil, sesame oil, maize oil, rapeseed oil, sunflower oil, cottonseed oil, jojoba oil, peanut oil, olive oil, and combinations thereof.

In one embodiment, the composition may comprise an oil selected from the group consisting of octyldodecanol, isododecanol, polyisobutene, polydecene, polyvinylpyrrolidone, mineral oil, and silicone oil.

The oil may be present in the composition, individually or in the aggregate, in an amount sufficient to form a gel with the polymeric gellant. The oil may be present individually or in the aggregate, in an amount between about 0.01% and about 25%, between about 0.1% and about 15%, between about 0.5% and about 10%, or between about 1% and about 8%, or between about 2% about 5% by weight of the composition.

The composition also typically comprises at least one film-forming agent. The film-forming polymer improves the wear of the composition, and can confer transfer-resistance to the make-up product. The film-forming agent may be any which is cosmetically acceptable for use around the eye. Examples include polymers such as polyethylene polymers, PVP, copolymers of PVP, ethylene vinyl acetate, dimethicone gum, C₁-C₆ alkyl (meth)acrylate polymer, polyacrylates, polymethacrylates, cellulose polymers, and resins such as trimethylsiloxysilicate.

Suitable polymeric film formers include, without limitation, acrylic polymers or copolymers, (meth)acrylates, alkyl (meth)acrylates, polyolefins, polyvinyls, polacrylates, polyurethanes, silicones, polyamides, polyethers, polyesters, fluoropolymers, polyethers, polyacetates, polycarbonates, polyamides, polyimides, rubbers, epoxies, formaldehyde resins, organosiloxanes, dimethicones, amodimethicones, dimethiconols, methicones, silicone acrylates, polyurethane silicones copolymers, cellulosics, polysaccharides, polyquaterniums, and the like. Suitable film formers include those listed in the Cosmetic Ingredient Dictionary and Handbook, 12th Edition (2008), the disclosure of which is hereby incorporated by reference.

Suitable silicone acrylate copolymers include those comprising a poly(alkyl)acrylate backbone and a dimethicone polymer grafted to an alkyl ester side chain, such as the commercially available film former Cyclopentasiloxane (and) Acrylates/Dimethicone Copolymer (KP-545, Shin-Etsu Chemical Co., Ltd) and Methyl Trimethicone (and) Acrylates/dimethicone Copolymer (KP-549, Shin-Etsu Chemical Co., Ltd.).

Additional suitable polymeric film formers include, without limitation, Amino Bispropyl Dimethicone, Aminopropyl Dimethicone, Amodimethicone, Amodimethicone Hydroxystearate, Behenoxy Dimethicone, C30-45 Alkyl Dimethicone, C24-28 Alkyl Dimethicone, C30-45 Alkyl Methicone, Cetearyl Methicone, Cetyl Dimethicone, Dimethicone, Dimethoxysilyl Ethylenediaminopropyl Dimethicone, Hexyl Methicone, Hydroxypropyldimethicone, Stearamidopropyl Dimethicone, Stearoxy Dimethicone, Stearyl Methicone, Stearyl Dimethicone and Vinyl Dimethicone. Particularly preferred are silicone polymers, including Methicone (as described by CTFA Monograph No. 1581, which is incorporated herein by reference), Dimethicones (as described by CTFA Monograph No. 840, which is incorporated herein by reference) and Amodimethicones as described by CTFA Monograph No. 189, which is incorporated herein by reference). In some embodiments, the film former comprises a hydrophilic film forming polymer, such as hydroxyethylcellulose or other cellulosics, PVP, and polyvinyl alcohol. Glyceryl rosinate may also be included as a film former. In another embodiment, the film former may comprise PVP/hexadecene copolymer. In some embodiments, the film former may comprise silicone film formers, such as cetyl hexacosyl dimethicone.

In one embodiment, the composition comprises a polyurethane film former, for example, those that are formed by reacting a di- or polyisocyanate with a diol and/or polyol), including for example, aqueous polyurethane dispersions. In one embodiment, the film former may comprise a copolymer of adipic acid, dicyclohexylmethane diisocyanate, ethylenediamine, Hexandiol, Neopentyl Glycol and sodium N-(2-aminoethyl)-3-aminoethanesulfonate monomers (INCI: Polyurethane-35; sold by Covestro as BAYCUSAN C1004). In another embodiment, the film former may comprise a copolymer of Hexanediol, Neopentyl Glycol, and Adipic Acid is reacted with hexamethylene diisocyanate, which may be further reacted with N-(2-aminoethyl)-3-aminoethanesulfonic acid and ethylenediamine (INCI: Polyurethane-34; sold by Covestro as BAYCUSAN C1000, and BAYCUSAN C1001). In another embodiment, the film former may comprise a copolymer of 1,4-Butanediol, ethylenediamine, hexamethylene diisocyanate, isophorone diisocyanate, and sodium N-(2-aminoethyl)-3-aminoethane sulfonate monomers (INCI: Polyurethane 32; sold by Covestro as BAYCUSAN C1003). In another embodiment, the film former may comprise waterborne polyurethane dispersion based on adipic acid, 1-6 hexandiol, neopentyl glycol, isophorone diisocyanate, isophorone diamine, N-(2-aminoethyl)-3-aminoethanesulphonicacid, sodium salt (INCI: Polyurethane 48; sold by Covestro as BAYCUSAN C1008).

In other embodiments, the film former may comprise AQUACOAT Gel (INCI: Polyurethane/PEG-6/PEG-90M), and/or ASCENA RC 880 (INCI: Polyurethane/PEG/TMHDI/Hexyldecanol/Octyldodecanol/PG/Aa).

In some embodiments, a polyurethane film former, such as polyurethane-35, is present in an amount from about 0.1-30% by weight (more typically from about 1-20% by weight) of the composition. In another embodiment, a polyurethane film former, such as polyurethane-35, is present in an amount from about 5-10%, or about 6-8% by weight of the composition.

If included, the film forming polymer may be present, individually or in the aggregate, in an amount of from about 0.01% to about 50%, or from about 0.05% to about 30%, or from about 0.1% to about 10% by weight of the composition. In some embodiments, the composition may comprise about 0.1% to about 5%, or about 0.5% to about 5%, relative to the total weight of the composition.

Particulates may optionally be included. Particulates may include any suitable pigments, lakes, fillers etc. Particulates will typically have a particle size between about 1 nm and about 1 mm. In some embodiments, at least 90% of the volume of particulates has a size (average diameter) greater than about 10 nm. In some embodiments, less than 10% of the total volume of particulates has a size greater than 100 microns. In one embodiment, particulates in the composition comprise those that are spherical and less than about 10 microns in diameter.

In one embodiment, the composition comprises particulates selected from the group consisting of carbon black, glass beads (e.g., borosilicate), iron oxide, silica, talc, and combinations thereof.

For purposes of the current invention, “pigments” shall be defined as organic pigments, inorganic pigments, lakes, pearlescent pigments, and or combinations thereof. Typically the compositions will include pigments to impart a desired color or effect. Color cosmetics, including mascaras, of the current invention may include black including various shades as well as additional known colors for mascaras. In certain, embodiments, the color white may be excluded from the colors of mascara available.

Examples of pigments are inorganic pigments, organic pigments, and/or lakes. Exemplary inorganic pigments include, but are not limited to, metal oxides and metal hydroxides such as magnesium oxide, magnesium hydroxide, calcium oxide, calcium hydroxides, aluminum oxide, aluminum hydroxide, iron oxides (α-Fe₂O₃, γ-Fe₂O₃, Fe₃O₄, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides and zinc oxides as well as composite oxides and composite hydroxides such as iron titanate, cobalt titanate and cobalt aluminate. Non-metal oxides also contemplated to be suitable are alumina and silica, ultramarine blue (i.e., sodium aluminum silicate containing sulfur), Prussian blue, manganese violet, bismuth oxychloride, talc, mica, sericite, magnesium carbonate, calcium carbonate, magnesium silicate, aluminum magnesium silicate, silica, titanated mica, iron oxide titanated mica, bismuth oxychloride, and the like. Organic pigments can include, but are not limited to, at least one of carbon black, carmine, phthalocyanine blue and green pigment, diarylide yellow and orange pigments, and azo-type red and yellow pigments such as toluidine red, litho red, naphthol red and brown pigments, and combinations thereof.

Lakes generally refer to a colorant prepared from a water-soluble organic dye, (e.g., D&C or FD&C) which has been precipitated onto an insoluble reactive or absorptive substratum or diluent. The term “D&C” as used herein means drug and cosmetic colorants that are approved for use in drugs and cosmetics by the FDA. The term “FD&C” as used herein means food, drug, and cosmetic colorants which are approved for use in foods, drugs, and cosmetics by the FDA. Certified D&C and FD&C colorants suitable for precipitation onto the insoluble reactive or absorptive stratum of lakes are listed in 21 C.F.R. §74.101 et seq. and include the FD&C colors Blue 1, Blue 2, Green 3, Orange B, Citrus Red 2, Red 3, Red 4, Red 40, Yellow 5, Yellow 6, Blue 1, Blue 2, Orange B, Citrus Red 2, and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Suitable lakes include, without limitation, those of red dyes from the monoazo, disazo, fluoran, xanthene, or indigoid families, such as Red 4, 6, 7, 17, 21, 22, 27, 28, 30, 31, 33, 34, 36, and Red 40; lakes of yellow pyrazole, monoazo, fluoran, xanthene, quinoline, dyes or salt thereof, such as Yellow 5, 6, 7, 8, 10, and 11; lakes of violet dyes including those from the anthroquinone family, such as Violet 2, as well as lakes of orange dyes, including Orange 4, 5, 10, 11, and the like. Suitable lakes of D&C and FD&C dyes are defined in 21 C.F.R. §82.51.

In addition to the foregoing, the compositions according to the invention may comprise additional pigments, and/or pearlescents. Inorganic pigments include without limitation titanium dioxide, zinc oxide, iron oxides, chromium oxide, ferric blue, mica, bismuth oxychloride, and titinated mica; organic pigments include barium, strontium, calcium or aluminum lakes, ultramarines, and carbon black. In certain, embodiments mascaras of the current invention exclude white pigments (e.g., titanium dioxide, zinc oxide, or calcium carbonate).

Suitable pearling pigments include without limitation bismuth oxychloride, guanine and titanium composite materials containing, as a titanium component, titanium dioxide, titanium lower oxides or titanium oxynitride, as disclosed in U.S. Pat. No. 5,340,569, the contents of which are hereby incorporated by reference. Other suitable pearlescent materials typically are pigments or layers of titanium dioxide on a substrate such as mica, polyethylene terephthalate, bismuth oxychloride, aluminum oxide, calcium borosilicate, synthetic flourophlogopite (synthetic mica), silica, acrylates copolymer, methyl methacrylate, and the like. Interference or pearl pigments may also be included. These are typically comprised of micas layered with about 50 to 300 nm films of TiO₂, Fe₂O₃, or Cr₂O₃ or the like. These include white nacreous materials, such as mica covered with titanium oxide or covered with bismuth oxychloride; and colored nacreous materials, such as titanium mica with iron oxides, titanium mica with ferric blue or chromium oxide, titanium mica with an organic pigment of the aforementioned type.

The pearlescent pigments can be chosen from white pearlescent pigments, such as mica covered with titanium or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride. Commercially available pearlescent pigments suitable for the current invention include, but are not limited to, MICAMIRA (Sandream Enterprises), SYNMIRA (Sandream Enterprises), GLASSMIRA (Sandream Enterprises), XIRONA (EMD Performance Chemicals), TIMIRON (EMD Performance Chemicals), COLORONA (EMD Performance Chemicals), RONASTAR (EMD Performance Chemicals), RONAFLAIR (EMD Performance Chemicals), REFLECKS (BASF), DUOCROME (BASF), and CHIONE (BASF).

The pigments may be optionally surface treated to, for example, make the particles more hydrophobic or more dispersible in a vehicle. The surface of the particles may, for example, be covalently or ionically bound to an organic molecule or silicon-based molecule or may be absorbed thereto, or the particle may be physically coated with a layer of material. The surface treatment compound may be attached to the particle through any suitable coupling agent, linker group, or functional group (e.g., silane, ester, ether, etc.). The compound may comprise a hydrophobic portion which may be selected from, for example, alkyl, aryl, allyl, vinyl, alkyl-aryl, aryl-alkyl, organosilicone, di-organosilicone, dimethicones, methicones, polyurethanes, silicone-polyurethanes, and fluoro- or perfluoro-derivatives thereof. Other hydrophobic modifiers include, but are not limited, lauroyl lysine, Isopropyl Titanium Triisostearate (ITT), ITT and Dimethicone (ITT/Dimethicone) cross-polymers, ITT and Amino Acid, ITT/Triethoxycaprylylsilane Crosspolymer, waxes (e.g., carnauba), fatty acids (e.g., stearates), HDI/Trimethylol Hexylactone Crosspolymer, PEG-8 Methyl Ether, Triethoxysilane, aloe, jojoba ester, lecithin, perfluoroalcohol phosphate, and Magnesium Myristate (MM). In other embodiments, the pigments or particulates may be surface treated with galactoarabinase or glyceryl rosinate. The pigments and particulates may be surface modified with, for example, fluoropolymers, to adjust one or more characteristics of the colorant as described in, for example, U.S. Pat. Nos. 6,471,950, 5,482,547, and 4,832,944, the contents of which are hereby incorporated by reference. In another embodiment, the pigments or particulates may be surface treated with Disodium Stearoyl Glutamate (and) Aluminum Dimyristate (and) Triethoxycaprylysilane. In one embodiment, the composition comprises a metal oxide surface treated with triethoxycaprylyl silane or trimethoxycaprylyl silane, including in amounts from about 0.1-10% by weight of the treated particulate (e.g., from about 1-5% by weight).

In some embodiments, the pigments include Iron Oxides, Black Oxide of Iron, Brown Iron Oxide, Iron Oxide Red 10-34-PC-2045, Pigment Black 11, Pigment Brown 6, Pigment Brown 7, Pigment Red 101, Pigment Red 102, Pigment Yellow 42, Pigment Yellow 43, Red Iron Oxide, Synthetic Iron Oxide, Yellow Iron Oxide, or carbon black. In some embodiments where carbon black is used as a pigment all or a portion thereof may be dispersed in a suitable synthetic wax.

The amount of all such pigments, individually or in the aggregate, is not particularly restricted. Typically, the particulates, including pigments and/or colorants, may comprise from about 0.01% to about 40% of the composition, from about 0.1% to about 20% by weight of the composition, or from about 1% to about 10% by weight of the composition. In certain embodiments, the composition will contain about 1%, 2.5%, 5%, 7.5%, 10%, 12.5%, or about 15% by weight pigments or other particulates. In embodiments incorporating carbon black as a pigment, the amount of carbon black incorporated may be about 0.005% to about 5%, or about 0.025% to about 1%, or about 1% to about 3% by weight of the composition. When the cosmetic composition is an emulsion, the pigments are added to the phase in which they are most compatible. For example pigments that have a hydrophobic treatment would be incorporated into the lipophilic phase, while pigments having a hydrophilic treatment would be in the aqueous phase. Pigments with silicone coatings would be incorporated into the silicone phase of a silicone-water emulsion.

In some embodiments, the total amount of particulates present in the composition is less than about 25% by weight, or less than about 20% by weight, or less than about 15% by weight, or less than about 12.5% by weight, or less than about 10% by weight, or less than 9% by weight, or less than about 8% by weight, or less than about 7% by weight, or less than about 6% by weight, or less than about 5% by weight, or less than about 4% by weight, or less than about 3% by weight, or less than about 2% by weight, or less than about 1% by weight, or less than about 0.5% by weight of the composition. In some embodiments, the compositions are free of particulates.

The compositions may, for example, comprise an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like having the appearance of a cream, gel or microemulsions. The emulsion may include an emulsifier, such as a nonionic, anionic or amphoteric surfactant, for example in an amount sufficient to stabilize the emulsion (e.g., 0.001-10% by weight).

The compounds suitable for use in the oil phase include any of the oils described herein. The oil-containing phase may be composed of a singular oil or mixtures of different oils. The oil phase may comprise from about 1-99% (or about 5-95%, or about 10-90%, or about 20-80%) by weight of the emulsion. The aqueous phase may comprise from about 1-99% or about 5-95%, or about 10-90%, or about 20-80% by weight of the emulsion.

The aqueous phase of the emulsion in one embodiment may have one or more organic compounds, including humectants (such as butylene glycol, propylene glycol, Methyl gluceth-20, and glycerin); other water-dispersible or water-soluble components including thickeners such as veegum or hydroxyalkyl cellulose; gelling agents, such as high MW polyacrylic acid, i.e. CARBOPOL 934; and mixtures thereof. In one embodiment, the aqueous phase may include a film forming polymer, for example an acrylate copolymer. In one embodiment, an acrylates copolymer is characterized as having a viscosity of about 25 cps in a 30% aqueous solution. The emulsion may have one or more emulsifiers capable of emulsifying the various components present in the composition.

Non-limiting emulsifiers include emulsifying waxes, emulsifying polyhydric alcohols, polyether polyols, polyethers, mono- or di-ester of polyols, ethylene glycol mono-stearates, glycerin mono-stearates, glycerin di-stearates, silicone-containing emulsifiers, soya sterols, fatty alcohols such as cetyl alcohol, acrylates, fatty acids such as stearic acid, fatty acid salts, and mixtures thereof. Emulsifiers may include soya sterol, cetyl alcohol, stearic acid, emulsifying wax, acrylates, silicone containing emulsifiers and mixtures thereof. Other specific emulsifiers that can be used in the composition of the present invention include, but are not limited to, one or more of the following: C₁₀₋₃₀ alkyl acrylate crosspolymer; Dimethicone PEG-7 isostearate, acrylamide copolymer; mineral oil; sorbitan esters; polyglyceryl-3-diisostearate; sorbitan monostearate, sorbitan tristearate, sorbitan sesquioleate, sorbitan monooleate; glycerol esters such as glycerol monostearate and glycerol monooleate; polyoxyethylene phenols such as polyoxyethylene octyl phenol and polyoxyethylene nonyl phenol; polyoxyethylene ethers such as polyoxyethylene cetyl ether and polyoxyethylene stearyl ether; polyoxyethylene glycol esters; polyoxyethylene sorbitan esters; dimethicone copolyols; polyglyceryl esters such as polyglyceryl-3-diisostearate; glyceryl laurate; Steareth-2, Steareth-10, and Steareth-20, to name a few. Additional emulsifiers are provided in the INCI Ingredient Dictionary and Handbook 11^{th} Edition 2006, the disclosure of which is hereby incorporated by reference in its entirety.

Water-in-silicone emulsions may be emulsified with a nonionic surfactant (emulsifier) such as, for example, polydiorganosiloxane-polyoxyalkylene block copolymers, including those described in U.S. Pat. No. 4,122,029, the disclosure of which is hereby incorporated by reference in its entirety. These emulsifiers generally comprise a polydiorganosiloxane backbone, typically polydimethylsiloxane, having side chains comprising -(EO)ₘ- and/or -(PO)ₙ- groups, where EO is ethyleneoxy and PO is 1,2-propyleneoxy, the side chains being typically capped or terminated with hydrogen or lower alkyl groups (e.g., C₁₋₆, typically C₁₋₃). Other suitable water-in-silicone emulsifiers are disclosed in U.S. Pat. No. 6,685,952, the disclosure of which is hereby incorporated by reference herein. Commercially available water-in-silicone emulsifiers include those available from Dow Corning under the trade designations 3225C and 5225C FORMULATION AID; SILICONE SF-1528 available from General Electric; ABIL EM 90 and EM 97, available from Goldschmidt Chemical Corporation (Hopewell, Va.); and the SILWET series of emulsifiers sold by OSI Specialties (Danbury, Conn.).

Examples of water-in-silicone emulsifiers include, but are not limited to, dimethicone PEG 10/15 crosspolymer, dimethicone copolyol, cetyl dimethicone copolyol, PEG-15 lauryl dimethicone crosspolymer, laurylmethicone crosspolymer, cyclomethicone and dimethicone copolyol, dimethicone copolyol (and) caprylic/capric triglycerides, polyglyceryl-4 isostearate (and) cetyl dimethicone copolyol (and) hexyl laurate, and dimethicone copolyol (and) cyclopentasiloxane. In one embodiment examples of water-in-silicone emulsifiers include, without limitation, PEG/PPG-18/18 dimethicone (trade name 5225C, Dow Corning), PEG/PPG-19/19 dimethicone (trade name BY25-337, Dow Corning), Cetyl PEG/PPG-10/1 dimethicone (trade name ABIL EM-90, Goldschmidt Chemical Corporation), PEG-12 dimethicone (trade name SF 1288, General Electric), lauryl PEG/PPG-18/18 methicone (trade name 5200 FORMULATION AID, Dow Corning), PEG-12 dimethicone crosspolymer (trade name 9010 and 9011 silicone elastomer blend, Dow Corning), PEG-10 dimethicone crosspolymer (trade name KSG-20, Shin-Etsu), dimethicone PEG-10/15 crosspolymer (trade name KSG-210, Shin-Etsu), and dimethicone PEG-7 isostearate.

The emulsifiers typically will be present in the composition in an amount effective to disperse the discontinuous phase into the continuous phase, typically from about 0.001% to about 10% by weight, in another embodiment in an amount from about 0.01% to about 5% by weight, and in a further embodiment in an amount below 1% by weight.

The aqueous phase of the emulsion may include one or more volatile solvents, including lower alcohols, such as ethanol, isopropanol, and the like. The volatile solvent may also be a cosmetically acceptable ester such as butyl acetate or ethyl acetate; ketones such as acetone or ethyl methyl ketone; or the like. The volatile solvents are generally present in an amount of 25% or less by weight of the composition. In other embodiments the volatile solvent is present in an amount of less than 15%, less than 10%, or less than 5% by weight of the composition. In another embodiment the compositions do not contain a volatile solvent.

The non-aqueous phase will typically comprise from about 10% to about 90%, about 30% to about 80%, or from about 50% to about 70% by weight, based on the total weight of the emulsion, and the aqueous phase will typically comprise from about 10% to about 90%, about 30% to about 80%, or from about 40% to about 70% by weight of the total emulsion. In one embodiment of the invention the mascara composition is a water-in-silicone emulsion in which the aqueous phase is from about 20% to about 60% by weight of the total composition and the non-aqueous silicone phase is from about 40% to 80% by weight of the total composition. In one embodiment of the invention the mascara composition is a water-in-oil or oil-in-water emulsion in which the aqueous phase is about 60% by weight of the total composition and the non-aqueous oil phase is about 40% by weight of the total composition.

The compositions may, for example, be anhydrous or may be substantially anhydrous. “Substantially anhydrous” as used herein means containing less than 5% by weight water. In other embodiments, the compositions will comprise less than about 2.5% by weight water, or less than 2% by weight water, or less than about 1% by weight water, or less than 0.25% by weight water. In some embodiments, the compositions may be anhydrous. The term “anhydrous” as used herein means that no water is added to the composition and that only that amount of moisture absorbed from the atmosphere will be present in the composition.

An anhydrous vehicle may include without limitation, vegetable oils; esters including emollient esters, such as octyl palmitate, isopropyl myristate and isopropyl palmitate; ethers such as dicapryl ether; fatty alcohols such as cetyl alcohol, stearyl alcohol octyldodecanol and behenyl alcohol; isoparaffins such as isooctane, isododecane and isohexadecane; silicone oils such as dimethicones, cyclic silicones, and polysiloxanes; hydrocarbon oils such as mineral oil, petrolatum, isoeicosane and polyisobutene; and the like. Suitable hydrophobic hydrocarbon oils may be saturated or unsaturated, have an aliphatic character and be straight or branched chained or contain alicyclic or aromatic rings. Such components may be present individually or in the aggregate, in an amount between about 0.01% to about 20% by weight of the composition.

Mascara compositions of the current invention may have a consistency of a liquid and/or viscous liquid. The hardness of the mascara may be measured by penetrating a probe into the composition. In particular, a texture analyzer (for example TA-XT2i from Rheo) equipped with a 2 mm needle probe may be used. The texture analyzer may be set to: Measurement Mode: Force in Compression; Test Speed: 1.0 mm/s; Distance: 5 mm; and Trigger Force: 5 g. The mascara compositions of the current invention may have a penetrating force of less than about 15 g and in other embodiments the penetrating force may be less than about 10 g. The hardness value may be between about 1 g and 15 g.

Additionally, the compositions of the current invention may exhibit a viscosity between about 250,000 centipoise and about 2,000,000 centipoise, in another embodiment between about 500,000 centipoise and about 1,750,000 centipoise; and about 750,000 centipoise and about 1,500,000 centipoise. The viscosity of the composition may be determined by using a Brookfield DV-E viscometer rotating at 4 rpms with a T-bar E spindle, at 25° C. In one embodiment, the composition is in the form of a liquid having a viscosity between about 250,000 cps and about 2,000,000 cps.

In an additional embodiment of the invention, a polyamide resin may provide additional structural integrity to the polymeric gellant. Polyamide resins are high molecular weight polymers which feature amide linkages along the molecular chain. These polymers contain monomers of amides joined by peptide bonds. They can occur both naturally and artificially. Such polymers are made through step growth polymerization or solid phase synthesis. In some cases, examples of polyamide resins are nylons and aramids. Due to their extreme durability and strength, polyamide resins are typically utilized in textiles, plastics and various automotive applications. In the composition of the present invention the polyamide resin also provides a degree of gloss or shine to the composition and adhesion to the target substrate.

In some embodiments, the polyamide resin may comprise Ethylenediamine Hydrogenated Dimer Dilinoleate Copolymer Bis-Di-C14-18 Alkyl Amide, however the invention is not limited to this polyamide resin. One skilled in the art will be able to select suitable polyamide resins and many suitable polymers are disclosed in the CTFA Handbook, 12′h Ed. 2008, the disclosure of which is hereby incorporated by reference. These include, without limitation, Polyamide-1, Polyamide-2, Polyamide-3, Ethylenediamine/Dimer Tallate Copolymer Bis-Hydrogenated Tallow Amide, Ethylenediamine/Stearyl Dimer Dilinoleate Copolymer, Ethylenediamine/Stearyl Dimer Tallate Copolymer, etc.

A polyamide resin, or a combination of compatible polyamide resins, may be present in an amount ranging from about 0.01% to about 25% by weight, about 1% to about 20% by weight, or about 5% to about 15% of the composition. In other embodiments, the compositions are free of polyamide resins.

The compositions of the invention may also comprise additional thickeners or viscosifying agents, such as, for example, a polysaccharide or non-polysaccharide thickener. For example, polymers and copolymers of acrylic acid, including Acrylates Copolymer (INCI) are contemplated to be suitable. The composition may also comprise silica, xanthan gum, CMC, acrylic acid polymers, bentone, triglycerides, aluminum stearate, C₁₈-C₃₆ acid glycol esters, glyceryl tribehenate, glycerol monostearate, alginates, carbomers, celluloses, hydrated magnesium and aluminium silicates, or calcium silicates, or the like. Oil-soluble rheology modifiers such as trihydroxystearin and/or 12-hydroxystearic acid may also be included.

In some embodiments, the compositions may comprise associative thickeners, such as polyurethane associative thickeners, including, for example, Bis-C16-20 isoalkoxy TMHDI/PEG-90 copolymer.

When present, thickeners and/or associative thickeners, may comprise, individually or in the aggregate, from about 0.01% to about 15% by weight of the composition, more typically from about 1% to about 5% by weight of the composition.

Compounds commonly used in the cosmetic arts for preventing or reducing fungal, bacterial, or microorganismal growth are also added to the composition of the disclosure. By including these compounds, the shelf life of the composition is lengthened. These anti-fungal and anti-microorganisms include but are not limited to methyl paraben, butyl paraben, sodium dehydroacetate, etc. The amounts of these ingredients that may be used within the inventive composition effectively reduce fungal, bacterial, and/or microorganismal growth without negatively affecting the components of the inventive composition or its desired effects.

The compositions of the invention may optionally comprise other active and inactive ingredients typically associated with the intended cosmetic or personal care products. Suitable other ingredients include, but are not limited to, amino acids, antioxidants, conditioners, chelating agents (e.g., sodium hexametaphosphate), pH adjusters (e.g., triethanolamine) colorants, emollients, emulsifiers, excipients, fillers, fragrances, gelling agents, humectants, minerals, moisturizers, photostabilizing agents (e.g., UV absorbers), sunscreens, preservatives (e.g., diazolidinyl urea), stabilizers, staining agents, surfactants, viscosity and/or rheology modifiers, vitamins, waxes and mixtures thereof. The additional components may be present individually or in the aggregate, in an amount between about 0.0001% and about 25%, between about 0.01% and about 15%, between about 0.1% and about 10%, or between about 1% and about 5% by weight of the composition.

All ingredients useful herein may be categorized or described by their postulated mode of action. However, it is to be understood that the ingredients can, in some instances, provide more than one cosmetic and/or therapeutic benefit or operate via more than one mode of action. Therefore, classifications herein are made for the sake of convenience and are not intended to limit an ingredient to the particularly stated application or applications listed.

It should be noted that although reference may be made throughout to mascara compositions, the inventive compositions and methods are applicable to any kind of cosmetic composition, including, for example, lipstick, lip color, lip gloss, nail polish, foundation, eye liner, and the like, as well as to any suitable personal care product, such as day creams or lotions, night creams or lotions, sunscreen lotions, sunscreen creams, sunscreen sprays or oils and other SPF products, moisturizers, salves, ointments, gels, body milks, artificial tanning compositions, facial masks, depilatories, shampoos, conditioners, hair masks, and the like.

Any of the compositions and ingredients therefor disclosed in U.S. Provisional Patent Application Ser. Nos. 61/789,975 and 61/790,104, both filed on Mar. 15, 2013, and in PCT/US14/27609 and PCT/US14/27692, both filed on Mar. 14, 2014, the entire contents of which are incorporated by reference herein for all purposes, are contemplated to be suitable for practice of the present invention.

The composition of the invention should be cosmetically or dermatologically acceptable, i.e., it should contain a non-toxic physiologically acceptable medium and should be able to be applied to the eyelashes of human beings. For the purposes of the invention, the expression “cosmetically acceptable” means a composition of pleasant appearance, odor, feel and taste.

Methods for styling keratin fibers are also provided. Any keratin fibers may be used, such as, for example, eyelashes, eyebrows, or hair on the head (scalp). The methods generally comprise applying to the surfaces of a keratin fiber a composition of the invention to form a moldable film on at least a portion of surfaces of the fibers (e.g., along at least a portion, or along a substantial portion, or along a majority of, or along substantially the entire length and/or circumference of the fiber).

The compositions may be applied to keratin fibers, for example with a brush, comb, or other suitable applicator (including, for example, any known mascara or cosmetics applicator). The compositions are applied to form a film or coating on the surface of individual fibers (e.g., lashes) along part or all of the length of the fiber. After the compositions have cured or partially cured, for example, by partial or complete evaporation of solvents and other volatiles, a dry film forms on the treated keratin fibers. This film is readily deformable and moldable due to its plastic, non-elastic nature.

A user may then apply a force to the treated keratin fibers (e.g., lashes) to style them into any desired configuration (i.e., a first configuration), such as, for example, a bent, curled, crimped, etc. configuration. When the force is removed, the lashes will remain substantially in the bent, curled, or crimped configuration. The treated keratin fibers may subsequently be re-molded into any number of additional configurations upon the application of subsequent forces, and after each of those forces is removed, the keratin fibers remain in each desired configuration. For example, the keratin fiber may be re-molded into a second desired configuration by applying a second force to the fibers, and so on. After the second force is removed, the keratin fibers remain substantially in the second desired configuration. In other words, the dried films, and consequently the treated fibers, are moldable and re-moldable after multiple applications.

Deformability, for example, may be characterized based on a test that displaces a 1-mm thick dry film 2 mm by a compression force at 25° C. The test may be performed on any suitable instrument, such as, for example, the Texture Analyzer described in Example 2.

Moldability, for example, may be characterized based on a test that measures the average of initial and sustained bend of treated lashes that have been bent or pushed, as described in Example 3.

The dried films may be characterized by a force on compression of less than 65 grams, or less than 60 grams, or less than 50 grams, or less than 40 grams, or less than 30 grams, or less than 20 grams, or less than 15 grams, when displaced by 2 mm at a constant force of 2 grams.

The keratin fibers may be molded by applying any suitable force to the fibers. The treated lashes may be styled with the application of forces less than, for example, 60 grams. The force may include any pressure applied to the fibers, such as, for example, by pressing, brushing, crimping, bending, twirling, squeezing, and so on, either with the use of one's fingers, or with any suitable instrument (e.g., a lash curler or crimper). In some embodiments, the step or steps of molding take place in the absence of added heat.

By way of illustration, after a film of a mascara of the invention has been applied to the eyelashes and dried or partially dried, the treated lashes can be molded into a curled configuration and remain curled after the force is removed. Subsequently, the lashes may be re-molded to a straightened configuration when a subsequent force is applied, and the lashes will remain straightened after the additional force is removed.

The compositions are also readily layerable, meaning, for example, that the force required to apply a second layer of the composition to a keratin fiber is less than 200%, or less than 175%, or less than 150%, or less than 125% of the amount of force required to apply a first layer of the composition to the keratin fiber. For example, layerability may be characterized based on a test that applies the composition with an instrument (such as the Texture Analyzer described in Example 4), and that measures the increase in force required to apply a second layer of the composition on top of a first dried layer, as described in Example 4.

The keratin fibers are also resistant to flaking after multiple applications. For example, in the case of a mascara composition, the mascara may be applied, or layered onto the lashes numerous times to refresh the color or to re-style the lashes, without substantial loss or flaking of the film.

In another embodiment, the invention relates to a method for coloring a human integument, including keratin fibers, comprising applying to the human integument a composition of the invention to form a film thereon. A human integument may include skin, lips, nails, hair, and other keratinous surfaces. As used herein, the term “keratinous surface” refers to keratin-containing portions of the human integumentary system, which includes, but is not limited to, skin, lips, hair (including hair of the scalp, eyelashes, eyebrows, facial hair, and body hair such as hair of the arms, legs, etc.), and nails (toenails, fingernails, cuticles, etc.) of mammalians, preferably humans.

In a further embodiment, the cosmetic compositions may impart color (e.g., black color) to the human integument (e.g., eyelashes).

A person skilled in the art will take care to select the optional additives and/or the amount thereof such that the advantageous properties of the composition according to the invention are not, or are not substantially, adversely affected by the envisaged addition. It is further understood that the other cosmetic ingredients and adjuvants introduced into the composition must be of a kind and quantity that are not detrimental to the advantageous effect which is sought herein according to the invention.

The following examples describe specific aspects of the compositions of the present invention to illustrate the invention and provide a description for those skilled in the art. The Examples should not be construed as limiting the invention as the examples merely provide specific methodology useful in the understanding and practice of the invention and its various aspects.

### EXAMPLES

### Example 1: Mascara Formulation

A mascara composition according to the current invention is provided in Table 3.

TABLE 3

| Ingredient | % by weight |
|---|---|
| Beeswax (Soft wax) | 4.8 |
| POE (20M) sorbitol beeswax (Soft wax) | 1 |
| Octyldodecanol (Oil) | 0.9 |
| Polymeric film former | 7.5 |
| Emulsifier | 8.1 |
| Chelator | 0.25 |
| pH adjuster | 2 |
| Particulates | 5 |
| Dimethicone film former | 4.4 |
| Associative thickener | 1 |
| Preservative | 0.5 |
| Polyurethane-35* | 15.6 |
| Demineralized water | q.s. |
| Butylene/Ethylene/Propylene copolymer (Gellant) | 0.4 |
| Polymeric shine agent | 1.5 |
| Thickener | 2.4 |

*40% by weight solids dispersed in water.

### Example 2: Deformability Test

A deformability test was used to assess the deformability of a dry film of the invention, and of a commercial mascara (Covergirl CLUMP CRUSHER). Films of the mascara compositions were prepared to be 1 mm thick, and were allowed to dry at 37° C. for 24 hours. The dry films were placed onto a film support rig that was fitted to a heavy duty platform of a TA-XT2 Texture Analyzer (Stable Micro Systems). The Texture Analyzer was equipped with a ¼ inch probe that was used to apply a downward target force of 2.0 g to the dry films, at a speed of 0.20 mm/sec, at a displacement distance of 2.0 mm. The force was then withdrawn from the films at 0.2 mm/sec, allowing the films to recover and exert an upward force on the probe. The force at compression (g) was measured, which represents how much downward force was required to displace the film by 2.0 mm. This force is a measure of deformability, or how much force it requires to deform or shape the film. A lower force at compression indicates that the film is more pliable, or easier to deform and shape.

The commercial mascara film required a force at compression of 83.66 g, whereas the inventive mascara film required a force at compression of only 11.17 g, indicating that the inventive mascara film was more deformable, or substantially softer and more pliable than the commercial mascara.

### Example 3: Moldability Test

A moldability test was used to assess the moldability of a dry film of the invention, and of a commercial mascara (Covergirl CLUMP CRUSHER). For each mascara sample, a bundle of three false eyelashes (Ardell 117) was attached to a Leneta card and placed on grid paper, and the reference spot on the grid (original coordinates) was noted while viewing through a microscope. Fourteen strokes of mascara were applied to the lashes, and after a 10-minute drying period, and additional 14 strokes of mascara were applied. The lashes were allowed to dry for 10 minutes. Moderate pressure was applied to “push” the lashes 14 times, and the distance (measured in mm) moved by the lashes (i.e., displacement of the tips from original coordinates) was recorded (initial bend). The lashes were left to sit for an hour, without an applied force. The displacement of the tips of the lashes from the original coordinates was again recorded (sustained bend). The average of the initial and sustained bend was calculated. The distance (mm) moved by the lashes is a measure of moldability, or the extent to which treated lashes can be configured or molded, and how well they stay in that configuration over time. More moldable mascara films “bend” a greater distance when pushed, and remain in that pushed configuration over time.

The commercial mascara film exhibited an average initial and sustained bend of 0.57 mm, whereas the inventive mascara film exhibited an average initial and sustained bend of 3.92 mm, indicating that the inventive mascara film is much more moldable than the commercial film. The inventive mascara film therefore behaves non-elastically. In some embodiments, the inventive mascaras will produce an average initial and sustained bend of greater than 1 mm, or greater than 2 mm, or greater than 3 mm.

### Example 4: Layerability Test

A layerability test was used to assess the force required to apply to lashes an initial layer and a second layer of a mascara of the invention, and of two commercial mascaras (Covergirl LASH BLOOM and Maybelline GREAT LASH). For each mascara sample, a TA-XT2 Texture Analyzer (Stable Micro Systems) was used to mechanically apply a first coat of mascara to a set of false lashes (Ardell 117). The force (g) required to apply the first layer of mascara was measured. After allowing the mascara film to dry, a second layer of the mascara was applied to the lashes, and the force required for application was measured. The percent change in force from the first application to the second application of mascara was calculated. The force of application is a measure of layerability, or how much force is required to layer a second application of mascara to lashes that have already been treated with mascara. A greater force indicates that it is more difficult to layer the mascara onto the lashes. The results are presented below in Table 4.

TABLE 4

| | Force on | Force on | |
|---|---|---|---|
| | First Coat | Second Coat | % |
| Sample | (g) | (g) | Increase |
| Inventive | 0.08 | 0.10 | 26.67 |
| Mascara | | | |
| Covergirl | 0.09 | 0.17 | 97.67 |
| LASH BLOOM | | | |
| Maybelline | 0.04 | 0.08 | 100 |
| GREAT LASH | | | |

The inventive mascara required the smallest increase in force for application of a second coat, with only a 26.67% increase. In contrast, for each of the commercial mascaras, the amount of force doubled, or nearly doubled when applying a second layer of mascara to the lashes. The inventive mascara therefore has better layerability compared to the commercial mascaras, as it was much easier to apply a second coat.

The invention described and claimed herein is not to be limited in scope by the specific embodiments herein disclosed since these embodiments are intended as illustrations of several aspects of the invention. Any equivalent embodiments are intended to be within the scope of this invention. Indeed, various modifications of the invention in addition to those shown and described therein will become apparent to those skilled in the art from the foregoing description. Such modifications are also intended to fall within the scope of the appended claims. All publications cited herein are incorporated by reference in their entirety.

1. A method for styling keratin fibers comprising:
(a) applying to keratin fibers a composition comprising an oil, a polymeric gellant for forming a gel with said oil, and a wax having a melting point below 70° C., which, upon evaporation of volatiles, forms a film on the surfaces of said keratin fibers characterized by a force on compression of less than 65 grams;
(b) molding said keratin fibers into a first configuration by applying a force thereto, wherein said keratin fibers remain in said first configuration after said force is removed.

2. The method according to claim 1, further comprising a step of re-molding said keratin fibers into a second configuration subsequent to said step of molding, wherein said keratin fibers remain in said second configuration after said second force is removed.

3. The method according to claim 1, wherein said applying step comprises a first step of applying a first layer of said composition to said keratin fibers, and a second step of applying a second layer of said composition on top of the first layer of said composition, wherein the force required to apply the second layer of the composition on top of the first layer of the composition on the keratin fibers is less than 50% greater than the force required to apply the first layer of the composition to the keratin fibers.

4. The method according to claim 1, wherein said composition further comprises from about 5% to about 30% by weight of polyurethane-35.

5. The method according to claim 1, wherein said step of molding occurs in the absence of applied heat.

6. The method according to claim 1, wherein said polymeric gellant comprises an ethylene mixed block copolymer of ethylene, propylene, and butylene having the INCI name butylene/ethylene/propylene copolymer.

7. The method according to claim 5, wherein said composition comprises:
(a) between about 0.1% and about 5% by weight of said ethylene mixed block copolymer of ethylene, propylene, and butylene;
(b) between about 0.1% and 10% of said oil capable of forming a gel with said ethylene mixed block copolymer;
(c) between about 7.5% and about 30% by weight of said wax having a melting point of less than 70° C.;
(d) between about 10% and about 20% by weight of a polymeric film former; and
(e) between about 0.5% and about 20% by weight of one or more particulates.

8. The method according to claim 1, wherein said composition is substantially free of waxes having a melting point of greater than 75° C.

9. The method according to claim 5, wherein said wax is selected from the group consisting of beeswax, paraffin wax, bleached beeswax, ozokerite, sorbitol beeswax, silicone wax, and PEG-modified beeswax.

10. The method according to claim 1, wherein said composition further comprises dibutyl laurolyl glutamide and/or dibutyl ethylhexanoyl glutamide.

11. The method according to claim 1, wherein said composition further comprises a pigment.

12. The method according to claim 1, wherein the keratin fibers are eye lashes.

13. The method according to claim 1, wherein the first configuration is a curled configuration.

14. The method according to claim 1, wherein the first configuration is a straight configuration.

15. A composition for application to keratin fibers comprising:
(a) between about 0.1% and about 5% by weight of a polymeric gellant comprising an ethylene mixed block copolymer of ethylene, propylene, and butylene;
(b) between about 0.1% and 10% of an oil capable of forming a gel with said polymeric gellant;
(c) between about 7.5% and about 30% by weight of a wax component comprising one or more waxes having a melting point of less than 70° C.°;
(d) between about 10% and about 20% by weight of a polymeric film former; and
(e) between about 0.5% and about 20% by weight of one or more particulates;
wherein said composition is capable of forming a film, which, upon evaporation of volatiles, is characterized by a force on compression of less than 65 grams.

16. The composition according to claim 15, wherein said one or more waxes is selected from the group consisting of beeswax, paraffin wax, bleached beeswax, ozokerite, sorbitol beeswax, silicone wax, and PEG-modified beeswax.

17. The composition according to claim 15, wherein said composition is substantially free of waxes having a melting point of greater than 75° C.

18. The composition according to claim 15, wherein said composition is free of waxes having a melting point of greater than 75° C.

19. The composition according to claim 15, wherein said composition further comprises dibutyl laurolyl glutamide and/or dibutyl ethylhexanoyl glutamide.

20. The composition according to claim 15, wherein said particulate further comprises a pigment.

21. The composition according to claim 15, wherein the composition is a mascara.

22. The composition according to claim 15, wherein the force required to apply a second layer of the composition over top of a first layer of the composition on keratin fibers is less than 50% greater than the force required to apply a first layer of the composition to the keratin fibers.
